(19) United States
(12) Patent Application Publication
(10) Pub. No.: US 2024/0366656 A1
(43) Pub. Date: Nov. 7, 2024

(54) Method of Treatment and Prophylaxis of Bacterial Infections

(71) Applicant: 3M INNOVATIVE PROPERTIES COMPANY, Saint Paul, MN (US)

(72) Inventors: Ranjani V. Parthasarathy, Woodbury, MN (US); Kevin D. Landgrebe, Woodbury, MN (US); Katie F. Wlaschin, St. Paul, MN (US)

(21) Appl. No.: 18/552,527

(22) PCT Filed: Apr. 5, 2022
(86) PCT No.: PCT/IB2022/053173

§ 371 Date: Sep. 26, 2023

Related U.S. Application Data
(60) Provisional application No. 63/173,878, filed on Apr. 12, 2021.

(51) Int. Cl. A61K 33/18; A61K 9/00; A61K 9/70; A61K 47/12; A61P 31/02; A61P 31/04

(52) U.S. Cl. CPC A61K 33/18; A61K 9/0043; A61K 9/006; A61K 9/7015; A61K 47/12; A61P 31/02; A61P 31/04

(57) ABSTRACT

A method, the method comprising applying a film-forming antiseptic composition to the nose and/or mouth of a mammalian subject; reducing a first bacterial load to a second bacterial load in the nasal cavity and/or the oral cavity of the mammalian subject over a duration of a treatment plan; and treating or preventing a bacterial infection in the mammalian subject.

### BACKGROUND

Bacteria can present problems including significant morbidity and loss of productivity in the population. Bacteremia refers to viable bacteria in the blood. Asymptomatic bacteremia can occur in normal daily activities such as conducting oral hygiene and after minor medical procedures. In a healthy person, these clinically benign infections are transient and cause no further sequelae. However, when immune response mechanisms fail or become overwhelmed, bacteremia can evolve into septicemia in which bacteria multiply in the blood and cause a number of clinical complications, including systemic inflammatory response syndrome (SIRS), sepsis, septic shock, and multiple organ dysfunction syndrome.

### SUMMARY

Thus, in one aspect, the present disclosure provides a method, the method comprising applying a film-forming antiseptic composition to the nose and/or mouth of a mammalian subject: reducing a first bacterial load to a second bacterial load in the nasal cavity and/or the oral cavity of the mammalian subject over a duration of a treatment plan; and treating or preventing a bacterial infection in the mammalian subject.

Various aspects and advantages of exemplary embodiments of the present disclosure have been summarized. The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### BRIEF DESCRIPTION OF THE DRAWINGS

FIG. 1 shows the mean weight gains/losses on days 0-3 for each animal in the four groups of 8 animals.

FIG. 2 shows mean colony counts in the lung on Day 3 for the four groups of 8 animals.

FIG. 3 shows colony counts in blood for days 1, 2 and 3 for 4 treatment groups

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized, and structural or logical changes may be made without departing from the scope of the present disclosure.

“Ambient temperature” refers to the temperature in the environment at which the method of the current invention is conducted. Typically, ambient temperature will be about 10° C. to about 30° C., and more particularly 15° C. to 25° C.

“Antiseptic” refers to a chemical agent that kills pathogenic and non-pathogenic microorganisms. Preferred antiseptics exhibit at least a 4 log reduction of both P. aeruginosa and S. aureus in 60 minutes from an initial inoculum of 1-3×10⁷ CFU/mL when tested in Mueller Hinton broth at 35° C. at a concentration of 0.25 wt.-% in a Rate of Kill assay using an appropriate neutralizer as described in “The Antimicrobial Activity in vitro of chlorhexidine, a mixture of isothiazolinones (Kathon CG) and cetyl trimethyl ammonium bromide (CTAB),” G. Nicoletti et al., Journal of Hospital Infection, 23, 87-111 (1993). Antiseptics generally interfere broadly with cellular metabolism and/or the cell envelope. Antiseptics are sometimes referred to as disinfectants, especially when used to treat hard surfaces.

“Available iodine” refers to iodine of the iodophor which is ultimately available to be released from the polymer as free iodine (I₂). It therefore includes free iodine in solution, diatomic iodine available from tri-iodide ions, as well as diatomic iodine held within a reservoir formed by the polymer structure. The available iodine does not include iodide ions. Available iodine is measured by thiosulfate titration in accordance with United States and British Pharmacopeia monographs.

“Effective amount” refers to the amount of the pharmaceutical composition and/or the enhancer component that, as a whole, provides an antibacterial activity that reduces, prevents, or eliminates one or more species of bacteria resulting in an acceptable level of the bacterial load.

Typically, an effective amount of antiseptic results in at least a 0.5 log reduction, and is desirably at least a 1 log reduction, and more preferably at least a 2-log reduction after a 30-minute contact time with the bacterium, preferably after only a 10-minute contact time with the bacterium, and most preferably after only a 5-minute min contact time with the bacterium.

It should be understood that in the compositions described herein, the concentrations or amounts of the components, when considered separately, may not prevent the growth of or inactivate and/or kill to an acceptable level, or may not prevent the growth of or inactivate or kill as broad a spectrum of bacteria, or may not inactivate or prevent the growth of or kill as fast; however, when used together such components provide an enhanced (preferably synergistic) antiviral activity and/or bacteriostatic and/or bactericidal activity (as compared to the same components used alone under the same conditions).

“Film-forming” refers to a composition that when allowed to dry under ambient conditions (e.g., 23° C. and 50% relative humidity (RH)) on in-tact skin forms a continuous layer that does not flake off after simple flexing of the tissue.

“Inoculation” refers to the act or process of introducing a pathogen such as a bacterium into a living organism. Inoculation can generally refer to exposing the mammalian subject to the pathogen directly (via introduction of the pathogen into the nose, for example) or indirectly or passively (via transmission from another animal or surface, for example). Inoculation occurs before the symptoms are observable in a mammalian subject.

“Mammalian subject” refers to humans, sheep, horses, cattle, pigs, dogs, cats, guinea pig, ferrets, rats, mice, or other mammal.

“Substantive” refers to an antiseptic composition (or a film-forming polymer in solution), that when applied to human skin as a uniform wet film in an amount of approximately 4 milligram per square centimeter (mg/cm²) clean dry skin on an inner forearm and allowed to thoroughly dry (e.g., at least 10 minutes at 23° C. C and 50% relative humidity), resists removal under running tap water at a temperature of about 23. degree. C. to about 24° C. C and a flow rate of about 2.4-2.5 liters/minute (L/min) falling from a height of 15 centimeters (cm) and striking the skin immediately above the dry composition (not directly on the dry composition) and then flowing over the dry composition for at least about 15 seconds.

“Symptom” refers to a physiological feature indicating a condition or disease. Symptoms of bacterial infection can include the general symptoms of inflammation, i.e., swelling, pain, chills, sweating, ocalized warming of tissue, localized redness of tissue, and other symptoms such as fever, nausea, sore throat, coughrunny nose, fever, aching muscles, headaches, chills, sweats, dry cough, fatigue, nasal congestion, sore throat, or combinations thereof.

“Treatment plan” refers to a sequence of treatments. “Treat” or “treatment” means to improve the condition of a subject relative to the affliction, typically in terms of clinical symptoms of the condition.

“Upper intake level” refers to safe intake level for iodine as established by Leung A M, Avram A M, Brenner A V, et al. Potential risks of excess iodine ingestion and exposure: statement by the American Thyroid Association Public Health Committee. Thyroid. 2015: 25 (2): 145-146.

It should be understood that (unless otherwise specified) the listed concentrations of all components are for “ready to use” or “as used” compositions. The compositions can be in a concentrated form. That is, certain embodiments of the compositions can be in the form of concentrates that would be diluted by the user with an appropriate vehicle; however, this is typically not convenient for the present application.

The terms “comprises” and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an infection means preventing, treating, or both treating and preventing further infections).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Film-forming antiseptic compositions can include those described in U.S. patent application No. 20180207122, and U.S. Pat. No. 8,808,722, which are incorporated by reference. Preferred antiseptic compositions are film-forming. For example, film forming polymers may retain the active antimicrobial component at the afflicted site and/or at a site where infection is initiated (e.g., the nares) for longer periods of time than compositions that do not contain a film former. This may be desirable for certain applications. For example, some film forming polymers may produce compositions that can not be washed off easily with water after being applied and dried. In at least one embodiment, the film-forming antiseptic composition can have a viscosity from 1000 to 10,000 centipoise.

In at least one embodiment, film-forming antiseptic compositions containing a broad spectrum antiseptic such as iodine or an iodophor, hydrogen peroxide, hypochlorite, chlorhexidine salts, poly hexamethylene biguanide, small molecule quaternary amines such as benzethonium chloride, methylbenzethonium chloride, benzalkonium chloride and octenidine, antimicrobial metals such as silver, triclosan, as well as combinations thereof, optionally with an enhancer component, can be used.

In at least one embodiment, the film-forming antiseptic composition can include a hydroxycarboxylic acid present at a concentration of at least 0.5% or 2.5 wt. %. The hydroxycarboxylic acid buffers of the present invention include preferably beta- and alpha-hydroxy acids (BHAs, AHAs, respectively, collectively referred to as hydroxy acids (HAs)), salts thereof, lactones thereof, and/or derivatives thereof. These may include mono-, di-, and tri functional carboxylic acids. Particularly preferred are HAs having 1 or 2 hydroxyl groups and 1 or 2 carboxylic acid groups. Suitable HAs include, but are not limited to, lactic acid, malic acid, citric acid, 2-hydroxybutanoic acid, 3-hydroxybutanoic acid, mandelic acid, gluconic acid, tartaric acid, salicylic acid, as well as derivatives thereof (e.g., compounds substituted with hydroxyls, phenyl groups, hydroxyphenyl groups, alkyl groups, halogens, as well as combinations thereof). Preferred HAs include lactic acid, malic acid, and citric acid. These acids may be in D, L, or DL form and may be present as free acid, lactone, or salts thereof. Other Suitable HAs are described in U.S. Pat. No. 5,665,776 (Yu et al.). The preferred HAs for use with iodine and in particular with povidone-iodine are lactic- and malic acid. Various combinations of hydroxycarboxylic acids can be used if desired.

The film-forming polymers may be nonionic, anionic, or cationic. They may also have pressure-sensitive adhesive properties. The film formers include both synthetic and natural polymers as well as derivatives of natural polymers. Preferred film-forming polymers are cationic. Preferred film-forming polymers are cationic polymers, particularly those that include side-chain functional amine groups. Examples of such groups include protonated tertiary amines, quaternary amines, amine oxides, and combinations thereof. Preferred such polymers are described in Applicants' Assignee's U.S. patent application Ser. No. 10/052, 10 15 25 30 35 40 45 50 55 60 65 12 158, filed on Jan. 16, 2002, entitled FILM-FORMING COMPOSITIONS AND METHODS, and published as U.S. Patent Publication No. 2003-01944.15 A1.

In at least one embodiment, the film-forming antiseptic composition can include at least 0.1 wt % of thickener—for example, cellulosics, such as quaternized cellulose, hydroxyethylcellulose, hydroxypropylmethylcellulose, methyl cellulose, cetyl hydroxyethyl cellulose, chitosans, carrageenan, guars or modified guar gums, xanthan gums or modified xanthan gums, gum Arabic, starches, pectin, dextran, alginates, pullulan, acrylates, polyvinyl alcohols, PVP, polymethyl vinyl ether, and polyurethanes. In some cases, the addition of these ingredients can result in an increase in viscosity of the solution.

In at least one embodiment, the film-forming antiseptic composition can include at least 0.1% of surfactants, for example, amphoteric surfactants, nonionic surfactants, and anionic surfactants, preferably amphoteric surfactants and non-ionic surfactants, for example, polyethyleneoxide extended sorbitan monoalkylates (polysorbates), polyalkoxylated alkanols, polyalkoxylated alkylphenols, polyglyceryl esters, polyalkoxylated esters, polyalkoxylated glycols, poloxamers, and alkyl polyglucosides.

Amphoteric surfactants can include those commercially available under the trade designations AMMONYX LO, AMMONYX LMDO, and AMMONYX CO, which are lauryldimethylamine oxide, laurylamidopropyldimethylamine oxide, and cetyl amine oxide, all from Stepan Company.

Preferably, film-forming antiseptic compositions that use iodine or iodophor (e.g., povidone-iodine (PVP-I)) are used. Some antiseptic compositions are also substantive. Examples of substantive antiseptic compositions are commercially available under the trade designation 3M Skin and Nasal Antiseptic by 3M (St Paul, MN). Additional suitable antiseptic compositions are commercially available under the trade designation Betadine from Aviro Health, Profend from PDI Healthcare, or Nozin from Global Life Technologies.

Suitable concentrations of iodophor can range from 0.1 to 10% by weight of antiseptic composition or from 0.1 wt. % to 2 wt. % antiseptic composition. In at least one embodiment, povidone-iodine can have a concentration of at least 1 wt. %. These concentrations are thought to be effective against various bacterial infections, for example, pneumonia, bacteremia, otitis media, and sinusitis, and combinations thereof.

In at least one embodiment, the film-forming antiseptic composition can be applied to the nose and/or mouth of a mammalian subject. The application of the film-forming antiseptic composition can include inserting a swab with the film-forming antiseptic composition impregnated therein at least 1 cm into the nasal cavity and spreading the film-forming antiseptic composition along the diameter of the nasal cavity.

In at least one embodiment, the film-forming antiseptic composition can be applied according to the treatment plan. In at least one embodiment, the film-forming antiseptic composition can reduce a first bacterial load to a second bacterial load in the nasal cavity and/or the oral cavity of the mammalian subject over a duration of the treatment plan. In at least one embodiment, the film-forming antiseptic composition can reduce the migration of the bacteria to locations selected from lung, blood, middle ear, sinuses, peritoneum, joint, meninges, and combinations thereof of the mammalian subject. The first bacterial load may be associated with an onset of symptoms for the bacterial infection. In some embodiments, the bacterial infection is not post-viral bacterial pneumonia, or bacterial pneumonia. In at least one embodiment, the film-forming antiseptic composition can treat or prevent a bacterial infection in the mammalian subject. In at least one embodiment, the film-forming antiseptic composition can treat or prevent a bacterial infection in an area in the mammalian subject distant from the nasal cavity and/or the oral cavity of the mammalian subject, for example, in the blood or lungs of the mammalian subject.

A first bacterial load at the onset of symptoms can be bacterial species-dependent and host species-dependent. For example, symptoms of bacterial infection can be present with a bacterial load of at least 10,000 colony forming units (CFU) per milliliter sample fluid. In at least one embodiment, the second bacterial load can be no greater than 100 colony forming units (CFU) per milliliter sample fluid.

The bacterial infection can be caused by bacteria, for example, antibiotic-susceptible- and antibiotic-resistant strains of Streptococcus spp., Staphylococcus spp., Haemophilus spp., Moraxella spp., Bacillus spp., Nocardia spp., Actinomyces spp., Neisseria spp., Klebsiella spp., Escherichia spp., Pseudomonas spp., Acinetobacter spp., Serratia spp., Mycobacterium spp., Legionella spp., and/or Mycoplasma spp. In at least one embodiment, the bacteria may be Streptococcus pneumoniae, Staphylococcus aureus, or methicillin-resistant Staphylococcus aureus.

The treatment plan can include options for treatment before (prophylaxis) inoculation with the bacteria, for example, applying a film-forming antiseptic composition to the nose and/or mouth of a mammalian subject before the first bacterial load is present in the mammalian subject. For example, in one treatment plan, the film-forming antiseptic composition can be applied to the mammalian subject up to 25, 20, 15, 10, 6, 5 or 3 hours before a first bacterial load is present in the mammalian subject.

In at least one embodiment, the treatment plan can include options of applying a film-forming antiseptic composition to the nose and/or mouth of a mammalian subject after a first bacterial load is present in the mammalian subject. For example, the film-forming antiseptic composition can be applied to the nose and/or mouth of a mammalian subject at least 4, 5, 10, 12, 18 or 24 hours after a first bacterial load is present in the mammalian subject.

In at least one embodiment, the treatment plan can occur over a duration. The duration can be at least 6 hours, at least 1 day, at least 3 days, at least 5 days, or at least 7 days. The duration should be no longer than 15 days, or no longer than 10 days.

The frequency of treatment can be no more than 6 times daily. For example, the frequency of treatment can be five times per day, four times per day, three times per day, twice per day, or once per day. The frequency of treatment can occur roughly evenly throughout the day. In at least one embodiment, the frequency of treatment can include attack dosages where treatment is concentrated during shorter intervals during the day. In at least one embodiment, the frequency of treatment will preferably be less than the upper intake level for the pharmaceutical composition. Further, the treatment plan will not allow the available iodine to surpass the toxicity for an adult human male for iodine (e.g., 2 g/day).

### Examples

These examples are merely for illustrative purposes and are not meant to limit the scope of the claims. All parts, percentages, ratios, etc. in the examples and the rest of the specification are by weight, unless noted otherwise. The symbol ‘g’ is used to represent the force of gravity. Solvents and other reagents used were obtained from Sigma-Aldrich Chemical Company, St. Louis, Missouri unless otherwise noted. “Nasal Prep” refers to 3M™ SKIN AND NASAL ANTISEPTIC, a product of the 3M Company, St. Paul, MN.

### Animals:

S. pneumoniae-negative C57BL/6 female mice, purchased from Jackson Laboratories and approximately 7-8 weeks old at the start of experiment, were acclimated for at least 5 days prior to use. Only animals that appeared healthy prior to the first administration of S. pneumoniae Serotype 2 were assigned to the study. The mice were randomized into study groups.

On Day 0, all study animals were infected intranasally with S. pneumoniae Serotype 2 (2×10⁶ CFU/50 μL/mouse).

### Infection (Day 0):

S. pneumoniae Preparation:

Brain heart infusion (BHI) broth (50 mL) was inoculated with a Microbank bead and incubated overnight at 37° C., 5% CO₂. Bacteria were centrifuged at 11,400×g for 15 minutes, resuspended in 1 mL of fresh serum broth (fetal bovine serum (FBS) plus BHI at a 1:5 ratio) and diluted with fresh serum broth such that a 0.7 optical density was reached at 500 nm. The culture was incubated at 37° C., 5% CO₂ for 4-5 hours or until an optical density of 1.6 at 500 nm was reached. Aliquots of the resulting culture were stored at −70° C. for up to 3 months to prevent any significant loss of viability.

Bacterial aliquots were thawed slowly at room temperature, centrifuged at 11,400×g for 15 minutes, and diluted to 2×10⁶ CFU/50 μL/mouse (4×10⁷ CFU/mL) in sterile 1X PBS.

S. pneumoniae Infection:

Mice were anesthetized with isoflurane and infected intranasally with S. pneumoniae (2×10⁶ CFU/50 μL/mouse) on Day 0. The actual number of viable bacteria used for infection was determined by plating dilutions of bacteria in duplicate on blood agar plates, both pre-infection (before animals were infected) and post infection (after all animals were infected). The results showed that the pre-infection and post-infection viable bacterial enumerations agreed with one another.

### Daily Observations

All mice were monitored once daily from Day −1 through Day 3. During observations, mice were monitored for clinical appearance, including body condition, coat appearance, posture, lethargy, and cyanotic paws and tails. Moribund animals displaying severe effects were discussed with the veterinarian and/or euthanized at the veterinarian's recommendation. Animals were monitored with an increased frequency (up to twice daily; at least 5 hours between observations) if adverse clinical signs were observed (including mortality of other animals on study). Abnormal findings were recorded as observed.

### Body Weight Measurements

All mice were weighed once daily from Day −1 through Day 3 for general health assessments. Body weights were recorded and reported.

### Terminal Procedure (Day 3)

On Day 3, animals were euthanized via CO₂ overdose, and terminal lung and body weights were recorded. Lungs were collected aseptically and processed as described below.

### Lung Collection for Bacterial Enumeration (Day 3)

Whole lung homogenates were prepared at 5% w/v in sterile 1X PBS and kept cold until plating.

### Bacterial Enumeration

Lung homogenate samples were diluted in sterile 1X PBS, and dilutions were plated, each in duplicate, on blood agar plates. Plates were incubated at 37° C., 5% CO₂ for 16-48 hours. Bacterial colonies were counted to determine the number of CFU.

### Results

Mice were treated according to one of the following conditions:
Treatment with saline 25 hours prior to infection
Treatment with Nasal Prep 25 hours prior to infection.
Treatment with Nasal Prep6 hours prior to infection and then daily.
Treatment with Nasal Prep 25 hours post-infection.

Mice in each treatment group were infected with 50 μl of S. pneumoniae diluted 1:2 (1.825×10⁶ CFU/50 μL/mouse).

Mice treated with saline had moderate weight loss (2/8 mice lost 10-15% bodyweight, 2/8 mice lost ≥15% bodyweight, and 1/8 mice lost ≥20% bodyweight) and 7/8 mice had clinical observations including ruffled fur and hunched posture on Days 1-3. Of these 7 mice, 5 were noted as having ruffled fur and hunched posture, while 2 were noted as having ruffled fur only. Mean group colony count in the lung was 3.32×10⁶ CFU/mL.

Mice treated with Nasal Prep25 hours prior to infection had moderate weight loss (5/8 mice lost 10-15% bodyweight, and 1/8 mice lost ≥20% bodyweight) and 5/8 mice had clinical observations including ruffled fur and hunched posture on Days 0-3. Of these 5 mice, 2 were noted as having ruffled fur and hunched posture, while 3 were noted as having either ruffled fur or hunched posture as a single observation. Mean group colony count in the lung was reduced compared to that for saline-treated mice (6.81×10³ CFU/mL).

Mice treated with Nasal Prep 6 hours prior to infection and then daily had moderate weight loss (1/8 mice lost 10-15% bodyweight, 3/8 mice lost ≥15% bodyweight, and 4/8 mice lost ≥20% bodyweight) and 8/8 mice had clinical observations including ruffled fur and hunched posture on Days 1-3. Of these 8 mice, 4 were noted as having ruffled fur and hunched posture, while 4 were noted as having hunched posture only. Mean group colony count in the lung was slightly reduced compared to saline-treated mice (8.09×10⁵ CFU/mL).

Mice treated with Nasal Prep 25 hours post-infection had moderate weight loss (2/8 mice lost 10-15% bodyweight, 1/8 mice lost ≥15% bodyweight, and 5/8 mice lost ≥20% bodyweight) and 8/8 mice had clinical observations of ruffled fur and hunched posture on Days 1-3. Hair loss was also noted for mice in each treatment group in this infection cohort but is considered incidental and unrelated to infection. Mean group colony count in the lung was slightly reduced compared to saline-treated mice (3.28×10⁵ CFU/mL)

### Tabular/Graphical Presentation of Results:

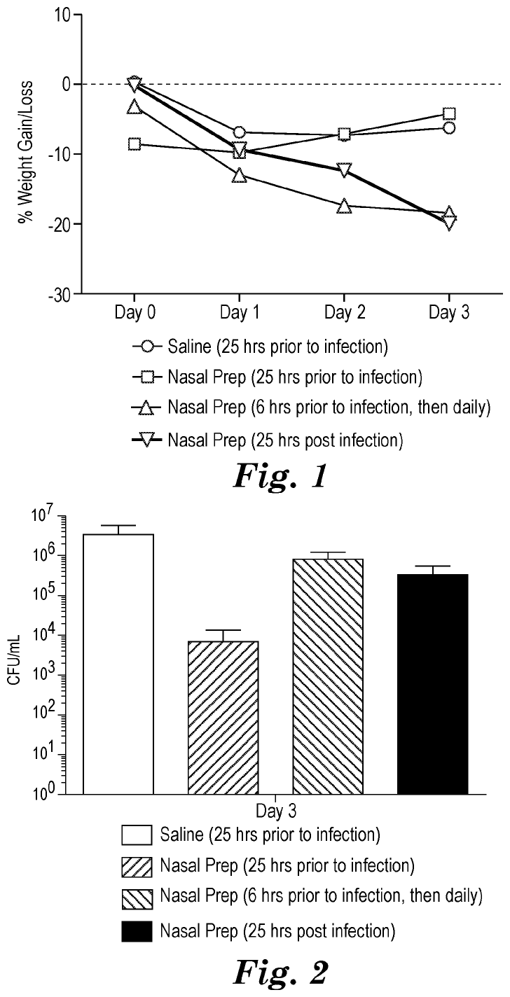

Mean weight gains/losses on days 0-3 for each animal in the four groups of 8 animals are shown in Table 1 and FIG. 1.

TABLE 1
Weight Gain/Loss.

| Infection | Treatment | Animal # | Day 0 | Day 1 | Day 2 | Day 3 |
|---|---|---|---|---|---|---|
| Streptococcus | Saline | 1* | 0.52% | −10.82% | −15.46% | −19.07% |
| pneumo | 25 hours | 2 | 0.00% | −10.64% | −13.83% | −18.62% |
| | | 3 | 1.10% | −10.44% | −13.74% | −10.99% |
| | | 4 | 2.27% | 1.14% | 6.25% | 9.09% |
| | | 5 | 1.69% | −6.78% | −10.73% | 1.69% |
| | | 6 | −2.08% | −3.12% | 4.17% | 5.73% |
| | | 7 | 1.10% | −1.66% | 2.76% | 5.52% |
| | | 8 | −1.78% | −12.43% | −17.75% | −23.08% |
| | Nasal Prep | 9 | −12.02% | −11.48% | −3.83% | −2.19% |
| | 25 hours | 10 | 0.58% | −6.36% | −6.36% | −10.98% |
| | prior to | 11 | −10.29% | −9.14% | −3.43% | 1.71% |
| | infection | 12 | −12.43% | −20.90% | −15.25% | −13.56% |
| | | 13 | −1.12% | −2.79% | −2.79% | −1.12% |
| | | 14 | −13.71% | −13.14% | −13.71% | −4.00% |
| | | 15 | −13.11% | −10.93% | −9.29% | −3.83% |
| | | 16 | −6.28% | −3.14% | −2.09% | 0.52% |
| | Nasal Prep | 17 | −3.70% | −10.05% | −16.40% | −10.58% |
| | 6 hours | 18 | −4.35% | −12.50% | −17.39% | −21.74% |
| | prior to | 19 | −3.87% | −16.02% | −19.34% | −24.86% |
| | infection, | 20 | −1.12% | −13.41% | −20.11% | −23.46% |
| | then daily | 21 | −3.37% | −12.92% | −19.10% | −17.42% |
| | | 22 | −1.08% | −6.99% | −10.75% | −5.38% |
| | | 23 | −3.17% | −17.46% | −19.58% | −23.81% |
| | | 24 | −4.19% | −14.14% | −16.23% | −19.90% |
| | Nasal Prep | 25 | 0.57% | −9.77% | −12.64% | −20.11% |
| | 25 hours | 26 | 1.21% | −10.91% | −13.94% | −22.42% |
| | post | 27 | 2.38% | −2.98% | −5.95% | −14.88% |
| | infection | 28 | −0.56% | −10.73% | −13.56% | −19.77% |
| | | 29 | −0.55% | −12.71% | −17.68% | −23.20% |
| | | 30 | 0.00% | −1.61% | 0.00% | −11.83% |
| | | 31 | −1.67% | −12.22% | −17.78% | −25.00% |
| | | 32 | −2.62% | 13.61% | −17.28% | −21.99% |

The results show that the saline-treated group and the group that was treated with Nasal Prep 25 hours prior to infection had the least weight loss. Further, the group that was treated with Nasal Prep 25 hours prior to infection had smaller weight losses in the animals that lost weight.

Mean colony counts in the lung on Day 3 for the four groups of 8 animals are shown in Table 2 and FIG. 2.

TABLE 2
Colony counts in the lung.

| | | | Animal | | Count | Count | | Group |
|---|---|---|---|---|---|---|---|---|
| Day | Infection | Group | # | Dilution | 1 | 2 | CFU/mL | Mean |
| Day 3 | 50 μl | Saline | 1* | | | | | 3.32E+06 |
| | 1:2 stock | 25 hrs | 2 | 1E+04 | 61 | 65 | 6.30E+06 | |
| | (Strep | prior to | 3 | 1E+00 | 96 | 44 | 7.00E+02 | |
| | Type 2) | infection | 4 | 1E+00 | 6 | 9 | 7.50E+01 | |
| | | | 5 | 1E+01 | 130 | 141 | 1.36E+04 | |
| | | | 6 | 1E+00 | 23 | 15 | 1.90E+02 | |
| | | | 7 | 1E+01 | 32 | 26 | 2.90E+03 | |
| | | | 8 | 1E+04 | 165 | 174 | 1.70E+07 | |
| | | Nasal Prep | 9 | 1E+02 | 52 | 57 | 5.45E+04 | 6.81E+03 |
| | | 25 hrs | 10 | 1E+00 | 2 | 1 | 1.50E+01 | |
| | | prior to | 11 | 1E+00 | 0 | 0 | 0.00E+00 | |
| | | infection | 12 | 1E+00 | 0 | 0 | 0.00E+00 | |
| | | | 13 | 1E+00 | 0 | 0 | 0.00E+00 | |
| | | | 14 | 1E+00 | 0 | 0 | 0.00E+00 | |
| | | | 15 | 1E+00 | 0 | 0 | 0.00E+00 | |
| | | | 16 | 1E+00 | 0 | 0 | 0.00E+00 | |
| | | Nasal Prep | 17 | 1E+00 | 0 | 0 | 0.00E+00 | 8.09E+05 |
| | | 6 hrs | 18 | 1E+03 | 221 | 231 | 2.26E+06 | |
| | | prior to | 19 | 1E+03 | 135 | 143 | 1.39E+06 | |
| | | infection, | 20 | 1E+03 | 270 | 294 | 2.82E+06 | |
| | | then daily | 21 | 1E+00 | 0 | 2 | 1.00E+01 | |
| | | | 22 | 1E+00 | 0 | 0 | 0.00E+00 | |
| | | | 23 | 1E+00 | 0 | 0 | 0.00E+00 | |
| | | | 24 | 1E+00 | 3 | 1 | 2.00E+01 | |
| | | Nasal Prep | 25 | 1E+00 | 0 | 0 | 0.00E+00 | 3.28E+05 |
| | | 25 hrs | 26 | 1E+00 | 0 | 0 | 0.00E+00 | |
| | | post | 27 | 1E+00 | 0 | 0 | 0.00E+00 | |
| | | infection | 28 | 1E+03 | 13 | 17 | 1.50E+05 | |
| | | | 29 | 1E+01 | 220 | 215 | 2.18E+04 | |
| | | | 30 | 1E+00 | 2 | 2 | 2.00E+01 | |
| | | | 31 | 1E+03 | 164 | 160 | 1.62E+06 | |
| | | | 32 | 1E+03 | 76 | 90 | 8.30E+05 | |

*Animal #1 died prior to lung harvest.

### Conclusion:

The results show that the lungs of the group of 8 animals treated with Nasal Prep 25 hours prior to infection had fewer than 1% of the bacteria that the saline-treated group (treated with saline 25 hours prior to infection) had in the lungs. The results show some reduction in lung counts for the other treatment groups compared with the saline-treated group.

### Animals

S. pneumoniae-negative C57BL/6 female mice, purchased from Jackson Laboratories and approximately 7-8 weeks old at the start of experiment, were acclimated for 4 days prior to use. Only animals that appeared healthy prior to the first administration of S. pneumoniae Serotype 2 were assigned to the study.

### Methodology

### Study Outline

This study examined the response to S. pneumoniae Serotype 2 infection in a C57BL/6 mouse model from Day 0 through Day 3. The study design required 66 female C57BL/6 mice (8 mice×2 infectious challenge cohorts×4 timepoints, plus 2 extra mice). Extra mice served as possible replacements for mice that were not healthy or were weight outliers prior to the experimental start date.

On Day 0, all study animals (and any applicable extra animals that serve as replacements) were infected intranasally with S. pneumoniae Serotype 2 (1.825×10⁶ CFU/mouse in 50 μL). Mice were dosed with Nasal Prep at a volume of 25 L/mouse (12.5 L/naris) at three timepoints/conditions (25 hours prior to infection, 6 hours prior to infection and daily thereafter, or 25 hours post-infection). Control mice were dosed with saline 25 hours prior to infection.

### Infection (Day 0)

S. pneumoniae Preparation

Inoculated brain heart infusion (BHI) broth (50 mL) was previously inoculated with a Microbank bead and incubated overnight at 37° C., 5% CO₂. Bacteria were centrifuged at 11,400×g for 15 minutes, resuspended in 1 mL of fresh serum broth (fetal bovine serum (FBS) plus BHI at a 1:5 ratio), and diluted with fresh serum broth such that a 0.7 optical density was reached at 500 nm. The culture was incubated at 37° C., 5% CO₂ for 4-5 hours or until an optical density of 1.6 at 500 nm was reached. Aliquots of the resulting culture are stored at −70° C. for up to 3 months to prevent any significant loss of viability.

Bacterial aliquots were thawed slowly at room temperature, centrifuged at 11,400×g for 15 minutes, and diluted to 1.825×10⁶ CFU/25 μL/mouse or 1.825×10⁶ CFU/50 μL/mouse (each equivalent to 1×10⁷ CFU/mL) in sterile 1X PBS.

S. pneumoniae Infection

Mice were anesthetized with isoflurane and infected intranasally with S. pneumoniae (1.825×10⁶ CFU/25 μL/mouse or 1.825×10⁶ CFU/50 μL/mouse) on Day 0. The actual number of viable bacteria used for infection was determined by plating dilutions of bacteria in duplicate on blood agar plates, both pre-infection (before animals were infected) and post infection (after all animals had been infected).

### Daily Observations

All mice were monitored once daily from Day −1 through Day 3. During observations, mice were monitored for clinical appearance, including body condition, coat appearance, posture, lethargy, and cyanotic paws and tails. Moribund animals displaying severe effects were discussed with the veterinarian and/or euthanized at the veterinarian's recommendation. Animals were monitored with an increased frequency (up to twice daily: at least 5 hours between observations) if adverse clinical signs were observed (including mortality of other animals on study). Abnormal findings were recorded as observed.

### Blood Collection for Bacterial Enumeration (Days 1, 2, and 3)

Non-terminal blood samples (75 μL) were collected via the saphenous vein (or tail vein or other IACUC-approved collection sites, if needed) on Days 1 and 2. Terminal blood collection samples were collected via cardiac puncture or via puncture of the inferior vena cava on Day 3. Blood samples were placed into pre-labeled blood collection tubes containing 0.675 mL sterile 1X PBS, immediately vortexed, and diluted and plated as described in the Bacterial Enumeration section.

### Terminal Procedures (Day 3)

On Day 3, animals were euthanized via CO₂ overdose and terminal blood collection samples were collected via cardiac puncture or via puncture of the inferior vena cava.

### Bacterial Enumeration

Blood homogenate samples were diluted in sterile 1X PBSand dilutions were plated, each in duplicate, on blood agar plates. Plates were incubated at 37° C., 5% CO₂ for 16-48 hours. Bacterial colonies were counted to determine the number of CFUs.

### Procedure and Results:

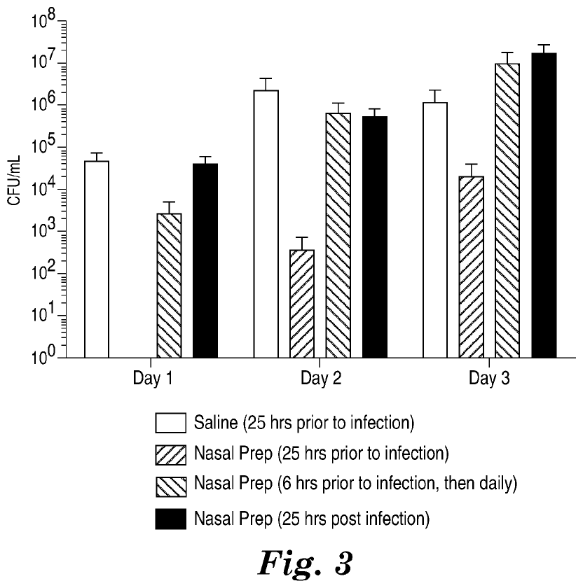

Individual animal data are presented in Table 3. Colony counts obtained from blood samples are presented in FIGS. 3 and 4.

Mice were treated with saline 25 hours prior to infection, Nasal Prep 25 hours prior to infection, Nasal Prep 6 hours prior to infection and then daily following infection or with Nasal Prep 25 hours post-infection. Mice in each treatment group were infected with 50 μL of S. pneumoniae diluted 1:2 (1.825×10⁶ CFU/50 μL/mouse)

In the S. pneumoniae 1.825×10⁶ CFU/50 μL/mouse cohort, mice treated with saline had moderate weight loss (2/8 mice lost 10-15% bodyweight, 2/8 mice lost ≥15% bodyweight, and 1/8 mice lost ≥20% bodyweight) and 7/8 mice had clinical observations including ruffled fur and hunched posture on Days 1-3. Of these 7 mice, 5 were noted as having ruffled fur and hunched posture, while 2 were noted as having ruffled fur only. Mean group colony count in blood was 4.69×10⁴ CFU/mL.

Mice treated with Nasal Prep 25 hours prior to infection had moderate weight loss (5/8 mice lost 10-15% bodyweight, and 1/8 mice lost ≥20% bodyweight) and 5/8 mice had clinical observations including ruffled fur and hunched posture on Days 0-3. Of these 5 mice, 2 were noted as having ruffled fur and hunched posture, while 3 were noted as having either ruffled fur or hunched posture as a single observation. Mean group colony count was reduced compared to saline-treated mice in blood (0.00 CFU/mL).

Mice treated with Nasal Prep 6 hours prior to infection and then daily following infection had moderate weight loss (1/8 mice lost 10-15% bodyweight, 3/8 mice lost ≥15% bodyweight, and 4/8 mice lost ≥20% bodyweight) and 8/8 mice had clinical observations including ruffled fur and hunched posture on Days 1-3. Of these 8 mice, 4 were noted as having ruffled fur and hunched posture, while 4 were noted as having hunched posture only. Mean group colony count was slightly reduced compared to saline-treated mice in the blood (2.64×10³ CFU/mL).

Mice treated with Nasal Prep 25 hours post-infection had moderate weight loss (2/8 mice lost 10-15% bodyweight, 1/8 mice lost ≥15% bodyweight, and 5/8 mice lost ≥20% bodyweight) and 8/8 mice had clinical observations of ruffled fur and hunched posture on Days 1-3. Hair loss was also noted for mice in each treatment group in this infection cohort but is considered incidental and unrelated to infection. Mean group colony count was slightly reduced compared to saline-treated mice in blood (3.97×10⁴ CFU/mL).

TABLE 3
Colony counts in the blood for each animal on days 1, 2, and 3.

| | | | Animal | | Count | Count | | Group |
|---|---|---|---|---|---|---|---|---|
| Day | Infection | Group | # | Dilution | 1 | 2 | CFU/mL | Mean |
| Day 1 | 50 μl | Saline | 1 | 1E+02 | 116 | 93 | 1.05E+05 | 4.68E+04 |
| | 1:2 stock | 25 hours | 2 | 1E+02 | 217 | 215 | 2.16E+05 | |
| | (Strep | prior to | 3 | 1E+01 | 0 | 0 | 0.00E+00 | |
| | Type 2) | infection | 4 | 1E+01 | 1 | 0 | 5.00E+01 | |
| | | | 5 | 1E+01 | 0 | 0 | 0.00E+00 | |
| | | | 6 | 1E+01 | 0 | 0 | 0.00E+00 | |
| | | | 7 | 1E+01 | 0 | 0 | 0.00E+00 | |
| | | | 8 | 1E+02 | 59 | 48 | 5.35E+04 | |
| | | Nasal Prep | 9 | 1E+01 | 0 | 0 | 0.00E+00 | 0.00E+00 |
| | | 25 hours | 10 | 1E+01 | 0 | 0 | 0.00E+00 | |
| | | prior to | 11 | 1E+01 | 0 | 0 | 0.00E+00 | |
| | | infection | 12 | 1E+01 | 0 | 0 | 0.00E+00 | |
| | | | 13 | 1E+01 | 0 | 0 | 0.00E+00 | |
| | | | 14 | 1E+01 | 0 | 0 | 0.00E+00 | |
| | | | 15 | 1E+01 | 0 | 0 | 0.00E+00 | |
| | | | 16 | 1E+01 | 0 | 0 | 0.00E+00 | |
| | | Nasal Prep | 17 | 1E+01 | 0 | 0 | 0.00E+00 | 2.64E+03 |
| | | 6 hours | 18 | 1E+01 | 196 | 204 | 2.00E+04 | |
| | | prior to | 19 | 1E+01 | 3 | 5 | 4.00E+02 | |
| | | infection, | 20 | 1E+01 | 5 | 9 | 7.00E+02 | |
| | | then daily | 21 | 1E+01 | 0 | 0 | 0.00E+00 | |
| | | | 22 | 1E+01 | 0 | 0 | 0.00E+00 | |
| | | | 23 | 1E+01 | 1 | 0 | 5.00E+01 | |
| | | | 24 | 1E+01 | 0 | 0 | 0.00E+00 | |
| | | Nasal Prep | 25 | 1E+01 | 0 | 0 | 0.00E+00 | 3.97E+04 |
| | | 25 hours | 26 | 1E+01 | 0 | 0 | 0.00E+00 | |
| | | post | 27 | 1E+01 | 0 | 0 | 0.00E+00 | |
| | | infection | 28 | 1E+02 | 131 | 138 | 1.35E+05 | |
| | | | 29 | 1E+02 | 85 | 103 | 9.40E+04 | |
| | | | 30 | 1E+01 | 0 | 0 | 0.00E+00 | |
| | | | 31 | 1E+01 | 4 | 11 | 7.50E+02 | |
| | | | 32 | 1E+02 | 89 | 87 | 8.80E+04 | |
| Day 2 | 50 μl | Saline | 1 | 1E+03 | 16 | 9 | 1.25E+05 | 2.20E+06 |
| | 1:2 stock | 25 hours | 2 | 1E+03 | 32 | 40 | 3.60E+05 | |
| | (Strep | prior to | 3 | 1E+01 | 0 | 0 | 0.00E+00 | |
| | Type 2) | infection | 4 | 1E+01 | 0 | 0 | 0.00E+00 | |
| | | | 5 | 1E+01 | 1 | 1 | 1.00E+02 | |
| | | | 6 | 1E+01 | 0 | 0 | 0.00E+00 | |
| | | | 7 | 1E+01 | 0 | 0 | 0.00E+00 | |
| | | | 8 | 1E+04 | 172 | 170 | 1.71E+07 | |
| | | Nasal Prep | 9 | 1E+01 | 0 | 0 | 0.00E+00 | 3.63E+02 |
| | | 25 hours | 10 | 1E+01 | 0 | 0 | 0.00E+00 | |
| | | prior to | 11 | 1E+01 | 0 | 0 | 0.00E+00 | |
| | | infection | 12 | 1E+01 | 33 | 25 | 2.90E+03 | |
| | | | 13 | 1E+01 | 0 | 0 | 0.00E+00 | |
| | | | 14 | 1E+01 | 0 | 0 | 0.00E+00 | |
| | | | 15 | 1E+01 | 0 | 0 | 0.00E+00 | |
| | | | 16 | 1E+01 | 0 | 0 | 0.00E+00 | |
| | | Nasal Prep | 17 | 1E+01 | 0 | 0 | 0.00E+00 | 6.37E+05 |
| | | 6 hours | 18 | 1E+03 | 114 | 129 | 1.22E+06 | |
| | | prior to | 19 | 1E+04 | 30 | 46 | 3.80E+06 | |
| | | infection, | 20 | 1E+02 | 80 | 81 | 8.05E+04 | |
| | | then daily | 21 | 1E+01 | 0 | 0 | 0.00E+00 | |
| | | | 22 | 1E+01 | 0 | 0 | 0.00E+00 | |
| | | | 23 | 1E+01 | 8 | 10 | 9.00E+02 | |
| | | | 24 | 1E+01 | 0 | 0 | 0.00E+00 | |
| | | Nasal Prep | 25 | 1E+01 | 0 | 0 | 0.00E+00 | 5.21E+05 |
| | | 25 hours | 26 | 1E+01 | 0 | 0 | 0.00E+00 | |
| | | post | 27 | 1E+01 | 0 | 0 | 0.00E+00 | |
| | | infection | 28 | 1E+03 | 190 | 185 | 1.88E+06 | |
| | | | 29 | 1E+02 | 123 | 140 | 1.32E+05 | |
| | | | 30 | 1E+01 | 0 | 0 | 0.00E+00 | |
| | | | 31 | 1E+03 | 24 | 13 | 1.85E+05 | |
| | | | 32 | 1E+03 | 206 | 190 | 1.98E+06 | |
| Day 3 | 50 μl | Saline | 1* | | | | | 1.13E+06 |
| | 1:2 stock | 25 hours | 2 | 1E+04 | 88 | 70 | 7.90E+06 | |
| | (Strep | prior to | 3 | 1E+01 | 0 | 0 | 0.00E+00 | |
| | Type 2) | infection | 4 | 1E+01 | 0 | 0 | 0.00E+00 | |
| | | | 5 | 1E+01 | 0 | 0 | 0.00E+00 | |
| | | | 6 | 1E+01 | 0 | 0 | 0.00E+00 | |
| | | | 7 | 1E+01 | 0 | 0 | 0.00E+00 | |
| | | | 8 | 1E+01 | 355 | 339 | 3.47E+04 | |
| | | Nasal Prep | 9 | 1E+01 | 0 | 0 | 0.00E+00 | 1.98E+04 |
| | | 25 hours | 10 | 1E+01 | 1 | 2 | 1.50E+02 | |
| | | prior to | 11 | 1E+01 | 0 | 0 | 0.00E+00 | |
| | | infection | 12 | 1E+02 | 162 | 155 | 1.59E+05 | |
| | | | 13 | 1E+01 | 0 | 0 | 0.00E+00 | |
| | | | 14 | 1E+01 | 0 | 0 | 0.00E+00 | |
| | | | 15 | 1E+01 | 0 | 0 | 0.00E+00 | |
| | | | 16 | 1E+01 | 0 | 0 | 0.00E+00 | |
| | | Nasal Prep | 17 | 1E+01 | 0 | 0 | 0.00E+00 | 9.58E+06 |
| | | 6 hours | 18 | 1E+03 | 165 | 190 | 1.78E+06 | |
| | | prior to | 19 | 1E+04 | 77 | 81 | 7.90E+06 | |
| | | infection, | 20 | 1E+05 | 61 | 73 | 6.70E+07 | |
| | | then daily | 21 | 1E+01 | 0 | 0 | 0.00E+00 | |
| | | | 22 | 1E+01 | 0 | 0 | 0.00E+00 | |
| | | | 23 | 1E+01 | 0 | 0 | 0.00E+00 | |
| | | | 24 | 1E+01 | 0 | 0 | 0.00E+00 | |
| | | Nasal Prep | 25 | 1E+01 | 3 | 0 | 1.50E+02 | 1.69E+07 |
| | | 25 hours | 26 | 1E+01 | 0 | 0 | 0.00E+00 | |
| | | post | 27 | 1E+01 | 0 | 0 | 0.00E+00 | |
| | | infection | 28 | 1E+05 | 31 | 51 | 4.10E+07 | |
| | | | 29 | 1E+02 | 82 | 79 | 8.05E+04 | |
| | | | 30 | 1E+01 | 0 | 0 | 0.00E+00 | |
| | | | 31 | 1E+04 | 138 | 175 | 1.57E+07 | |
| | | | 32 | 1E+05 | 80 | 77 | 7.85E+07 | |

*Animal #1 died prior to harvest.

### Conclusion:

The results show that the group of 8 animals had significantly lower bacteria in blood when Nasal Prep was used 25 hours prior to infection relative to saline. The results show some reduction in blood counts for the other treatment groups compared with the saline-treated group. For the other treatment group (6 hours prior to infection, then daily), the use of Nasal Prep did not show a significant decrease in bacterial counts in blood. Using Nasal Prep 25 hours post infection may be too late to prevent bacteremia.

In conclusion, treatment with Nasal Prep 25 hours prior to infection resulted in the overall greatest reduction of weight loss, clinical observations, and S. pneumoniae colonies in blood following infection when compared to saline control.

All references and publications cited herein are expressly incorporated herein by reference in their entirety into this disclosure. Illustrative embodiments of this invention are discussed, and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A method, the method comprising
applying a film-forming antiseptic composition to the nose and/or mouth of a mammalian subject;
reducing a first bacterial load to a second bacterial load in the nasal cavity and/or the oral cavity of the mammalian subject over a duration of a treatment plan; and
treating or preventing a bacterial infection in the mammalian subject;
wherein the duration of the treatment plan is at least 3 days and no longer than 15 days; and
wherein the frequency of the treatment is no more than 6 times per day.

2. The method of claim 1, further comprising reducing the migration of the bacteria to locations selected from lung, blood, middle ear, sinuses, peritoneum, joint, meninges, and combinations thereof of the mammalian subject.

3. The method of claim 1, wherein the first bacterial load is associated with an onset of symptoms for the bacterial infection.

4. The method of claim 1, wherein the bacterial infection comprises pneumonia, bacteremia, otitis media, and sinusitis.

5. The method of claim 1, wherein the bacterial infection is not post-viral bacterial pneumonia or bacterial pneumonia.

6. The method of claim 1, wherein the film-forming antiseptic composition comprises iodine, iodophor, or combinations thereof.

7. The method of claim 1, wherein the iodine or iodophor is present in a sufficient concentration to provide an available iodine concentration of 0.1 wt.-% to 2 wt.-%.

8. The method of claim 1, wherein the iodophor is povidone-iodine (PVP-I) with a concentration of at least 1 wt. %.

9. The method of claim 1, wherein the treatment plan comprises applying the film-forming antiseptic composition at a frequency of no more than 6 times daily.

10. The method of claim 1, wherein the treatment plan comprises applying the film-forming antiseptic composition at a frequency of once daily.

11. The method of claim 1, wherein applying the film-forming antiseptic composition to the nose and/or mouth of a mammalian subject occurs before the first bacterial load is present in the mammalian subject.

12. The method of any of claim 1, wherein applying the film-forming antiseptic composition to the nose and/or mouth of a mammalian subject occurs up to 25 hours before a first bacterial load is present in the mammalian subject.

13. The method of claim 1, wherein applying the film-forming antiseptic composition to the nose and/or mouth of a mammalian subject occurs up to 6 hours before a first bacterial load is present in the mammalian subject.

14. The method of claim 1, wherein applying the film-forming antiseptic composition to the nose and/or mouth of a mammalian subject occurs after a first bacterial load is present in the mammalian subject.

15. The method of claim 1, applying the film-forming antiseptic composition to the nose and/or mouth of a mammalian subject at least 4 hours after a first bacterial load is present in the mammalian subject.

16. The method of claim 1, applying the film-forming antiseptic composition to the nose and/or mouth of a mammalian subject at least 24 hours after a first bacterial load is present in the mammalian subject.

17. (canceled)

18. The method of claim 1, wherein the bacteria comprise antibiotic-susceptible- and antibiotic-resistant strains of Streptococcus spp., Staphylococcus spp., Haemophilus spp. Moraxella spp., Bacillus spp., Nocardia spp., Actinomyces spp., Neisseria spp., Klebsiella spp., Escherichia spp., Pseudomonas spp., Acinetobacter spp., Serratia spp., Mycobacterium spp., Legionella spp., and/or Mycoplasma spp

19. The method of claim 1, wherein the bacteria comprise Streptococcus pneumoniae, Staphylococcus aureus, or methicillin-resistant Staphylococcus aureus.

20. The method of claim 1, wherein the first bacterial load is at least 10,000 colony forming units (CFU) per milliliter sample fluid.

21. The method of claim 1, wherein the second bacterial load is no greater than 100 colony forming units (CFU) per milliliter sample fluid.

22. The method of claim 1, wherein the film-forming antiseptic composition comprises a hydroxycarboxylic acid present at a concentration of at least 2.5 wt.-%, a thickener present at a concentration of at least 0.25 wt.-%, and surfactants present at a concentration of at least 0.1 wt.-%.
